(19) United States
(12) Patent Application Publication
(10) Pub. No.: US 2016/0220636 A1
(43) Pub. Date: Aug. 4, 2016

(54) METHOD OF TREATING AUTO-IMMUNE OR INFLAMMATORY DISEASES USING B7 FAMILY PROTEINS OF DERIVATIVES THEREOF

(71) Applicants: SARAH BACUS, Hinsdale, IL (US); DIARMUID MORAN, Chicago, IL (US)

(72) Inventors: SARAH BACUS, Hinsdale, IL (US); DIARMUID MORAN, Chicago, IL (US)

(21) Appl. No.: 15/009,036

(22) Filed: Jan. 28, 2016

Related U.S. Application Data
(60) Provisional application No. 62/109,305, filed on Jan. 29, 2015.

(51) Int. Cl. A61K 38/17; A61K 9/00

(52) U.S. Cl. CPC A61K 38/1774; A61K 9/0014

(57) ABSTRACT

The present invention provides methods of treating auto-immune or inflammatory diseases comprising administering an amount of a B7 family protein or derivative thereof to a subject in need thereof.

### BACKGROUND OF THE INVENTION

This application claims the benefit of U.S. patent application Ser. No. 62/109,305, filed Jan. 29, 2015, the contents of which are incorporated herein by reference.

### FIELD OF THE INVENTION

The present invention is directed to methods of treating auto-immune or inflammatory diseases comprising administering to a subject in need thereof an amount of B7 family proteins or derivatives thereof to a subject in need thereof.

### BACKGROUND ART

B7 family proteins are structurally related, cell-surface protein ligands, which bind to receptors on lymphocytes that regulate immune responses. Activation of T and B lymphocytes is initiated by engagement of cell-surface, antigen-specific T-cell receptors or B-cell receptors, but additional signals delivered simultaneously by B7 ligands determine the ultimate immune response. These ‘costimulatory’ or ‘coinhibitory’ signals are delivered by B7 ligands through the CD28 family of receptors on lymphocytes. Interaction of B7-family members with costimulatory receptors augments immune responses, and interaction with coinhibitory receptors attenuates immune responses. There are multiple known members of the family, including but not limited to B7.1 (CD80), B7.2 (CD86), inducible costimulator ligand (ICOS-L), programmed death-1 ligand (PD-L1), programmed death-2 ligand (PD-L2), B7-H3, and B7-H4.

Programmed death-ligand 1 (PD-L1) is a 40 kDa type 1 transmembrane protein that has been speculated to play a major role in suppressing the immune system during particular events such as pregnancy, tissue allografts, auto-immune disease and other disease states such as hepatitis. Normally the immune system reacts to foreign antigens where there is some accumulation in the lymph nodes or spleen which triggers a proliferation of antigen-specific CD8+ T cell. The formation of PD-1 receptor/PD-Ll or B7.1 receptor/PD-L1 ligand complex transmits an inhibitory signal which reduces the proliferation of these CD8+ T cells at the lymph nodes and supplementary to that PD-1 is also able to control the accumulation of foreign antigen specific T cells in the lymph nodes through apoptosis which is further mediated by a lower regulation of the gene Bcl-2. PD-L1 binds to its receptor, PD-1, found on activated T cells, B cells, and myeloid cells, to modulate activation or inhibition. The affinity between PD-L1 and PD-1, as defined by the dissociation constant Kd, is 770 nM.

Auto-immune or inflammatory diseases arise from an inappropriate immune response of the body against substances and tissues normally present in the body. This may be restricted to certain organs or involve a particular tissue in different places. The treatment of auto-immune diseases is typically with immunosuppression medication that decreases the immune response.

PD-1/PD-L1 interaction is implicated in auto-immunity from several lines of evidence. NOD mice, an animal model for auto-immunity in that they exhibit a susceptibility to spontaneous development of type I diabetes and other auto-immune diseases, have been shown to have precipitated onset of diabetes from blockade of PD-1 or PD-L1 (but not PD-L2).

In humans, PD-L1 was found to have altered expression in pediatric patients with systemic lupus erythematosus (SLE). Studying isolated peripheral blood mononuclear cell (PBMC) from healthy children, immature myeloid dendritic cells and monocytes expressed little PD-L1 at initial isolation, but spontaneously up-regulated PD-L1 by 24 hours. In contrast, both mDC and monocytes from patients with active SLE failed to upregulate PD-L1 over a 5 day time course, expressing this protein only during disease remissions. This suggests one mechanism whereby peripheral tolerance is lost in SLE.

The present invention provides a method for treating auto-immune or inflammatory diseases through the administration of B7 family proteins or derivatives thereof to a subject in need thereof. In some embodiments, a treatment is directed to an auto-immune and inflammatory skin disease, such as vitiligo, psoriasis, or atopic dermatitis. In some embodiments, the B7 family protein comprises programmed death ligand-1 or derivative thereof. In some embodiments, the B7 family proteins or derivatives thereof are administered topically.

### SUMMARY OF THE INVENTION

In one embodiment, present invention is directed to methods of treating auto-immune or inflammatory diseases comprising administering to a subject in need thereof an amount of B7 family proteins or derivatives thereof to a subject in need thereof.

In some embodiments, the auto-immune or inflammatory disease treated is an auto-immune or inflammatory skin disease, including, but not limited to, vitiligo, psoriasis, or atopic dermatitis.

In some embodiments, a B7 family protein comprises programmed death ligand-1 or derivatives thereof.

In some embodiments, the present invention comprises topically administering a B7 family protein or derivative thereof.

In some embodiments, a B7 family proteins includes modified proteins to improve parameters such as dermal penetration, therapeutic efficacy, storage stability, conformational stability, or bioavailability. In some embodiments, modifications may include myristoylation, palmitoylation, pegylation, or sequence re-arrangement.

### DETAILED DESCRIPTION OF THE INVENTION

The present invention provides methods of treating auto-immune or inflammatory diseases comprising administering to a subject in need thereof an amount of B7 family proteins or derivatives thereof to a subject in need thereof. In some embodiments, a treatment is directed to an auto-immune and inflammatory skin disease, including, but not limited to, vitiligo, psoriasis, or atopic dermatitis. In some embodiments, the B7 family protein comprises programmed death ligand-1 or derivative thereof. In some embodiments, the B7 family proteins or derivatives thereof are administered topically.

As used here, the terms “treatment” or “treating” refer to the alleviation, amelioration, combating, management, or prevention of symptoms or causes of a medical condition, disorder, injury, or disease. In some embodiments, “treatment” or “treating” refers to an active or curative treatment, designed to cure a disease. In some embodiments, “treatment” or “treating” refers to palliative treatment, directed to relieve pain and distress. In some embodiments, “treatment” or “treating” refers to causal treatment, which is directed at the cause of a disease or disorder. In some embodiments, “treatment” or “treating” refers to expectant treatment, directed toward relief of untoward symptoms.

As used here, the term “administering” or “administration” refers to the introduction of a therapeutically active compound to the body by acceptable routes known to those of skill in the art including, but not limited to transdermally, cutaneously, topically, nasally, rectally, vaginally, sublingually, buccally, subcutaneously, intrathecally, intramuscularly, intraveneously, orally, or through application to the eye, ear, or to the lungs through inhalation or nebulization.

As used here, the term “auto-immune or inflammatory diseases” refers to diseases, disorders, or other conditions arising from a body's own abnormal immune response against cells, substances, tissues or organs normally present in the body. In some embodiments, an auto-immune diseases includes multiple sclerosis, where the immune system is suspected to attack and damage the body's own myelin insulation of the nerve cells. In other embodiments, an auto-immune disease includes rheumatoid arthritis, wherein the immune system is suspected to attack and damage the tissue of the joints. In other embodiments, an auto-immune disease includes vitiligo, wherein the immune system is suspected to attack and damage melanin pigmentation cells. In other embodiments, the auto-immune or inflammatory disease is idiopathic, wherein the cause or target of the abnormal immune or inflammatory response is unknown or not understood, such as idiopathic inflammatory myopathy. In other embodiments an auto-immune or inflammatory disease includes, but is not limited to, psoriasis, topic dermatitis, systemic lupus, ulcerative colitis, scleroderma, sarcoidosis, psoriatic arthritis, primary biliary cirrhosis, pernicious anaemia, pemphigus vulgaris, Guillain-Barre syndrome, Graves' disease, endometriosis, eczema, diabetes mellitus type-1, dermatomyositis, Crohn's disease, celiac disease, auto-immune hepatitis, or ankylosing spondylitis.

As used here, the term “auto-immune or inflammatory skin diseases” refers to a subset of auto-immune or inflammatory diseases originating in, manifesting symptoms in, or otherwise affecting the cells, tissues, or substances of the integumentary system, comprising the epidermis, dermis, hypodermis, and associated appendages, such as hair follicles and fingernails. In some embodiments, an auto-immune or inflammatory skin disease includes, but is not limited to, psoriasis, vitiligo, atopic dermatitis, epidermolysis bullosa acquista, pemphigus vulgaris, IgA-mediated bullous dermatoses, systemic lupus erythematosus, alopecia areata, porphyria, or scleroderma.

As used here, the term “B7 family proteins or derivatives thereof” refers to any of the structurally related, cell-surface protein ligands found on activated antigen presenting cells (APC) that, when paired with either a CD28 or CD 152 (CTLA-4) surface protein on a T-cell, modulate T-cell activity, or derivative polypeptides resulting from the modification of such B7 family proteins. Known B7 family proteins include, but are not limited to, B7-1 (CD80), B7-2 (CD86), B7-DC (PDCD1LG2, PD-L2, or CD273), B7-H1 (PD-L1, CD274). These known proteins or naturally occurring variants thereof can be modified using known techniques, creating derivative polypeptides.

As used here, “derivatives” refers full-length, partial, cross-linked, chimeric, denatured, augmented, genetically-modified, chemically-modified, or other variant polypeptides that result from the modification of B7 family proteins. In some embodiments, derivatives include modified or unmodified ribosomal peptides, non-ribosomal peptides, peptones, or peptide fragments. In some embodiments, derivatives include any biologically or therapeutically active peptide fragments of B7 family proteins or derivatives thereof, which may be further modified to increase therapeutic efficacy.

As used here, the term “modification” in relation to B7 family proteins or derivatives thereof means an augmenting, altering, crosslinking, cleavage, fragmenting, denaturing, or otherwise changing naturally-occurring or synthetic polypeptide chains. An example of such a modification includes myristoylation, the irreversible covalent addition of a myristoyl group to the alpha-amino group of an N-terminal glycine residue. Another example of such a modification includes palmitoylation, the typically reversible covalent attachment of fatty acids (such as palmitic acid) to a protein. Another example of such a modification is pegylation, the covalent attachment of polyethylene glycol (PEG) polymer to a protein or other molecule. Other such modifications known in the art include but are not limited to sequence rearrangement, recombination, conjugation, or fragmentation.

While not being bound to any particular theory, the present invention, the present invention provides a novel route of immunosuppression for the treatment of auto-immune or inflammatory diseases through B7 family proteins or derivatives thereof interaction with co-inhibitory receptors on immune cells to attenuate abnormal immune responses. Auto-immune or inflammatory diseases have various known and unknown root causes, but all share an abnormal immune system response to substances, cells, or tissues that are usually present and tolerated by healthy immune systems.

B7 family proteins consists of structurally related, cell-surface protein ligands, which bind to receptors on lymphocytes that regulate immune responses. Activation of T and B lymphocytes is initiated by engagement of cell-surface, antigen-specific T-cell receptors or B-cell receptors, but additional signals delivered simultaneously by B7 ligands determine the ultimate immune response. These ‘costimulatory’ or ‘coinhibitory’ signals are delivered by B7 ligands through the CD28 family of receptors on lymphocytes. Interaction of B7-family members with costimulatory receptors augments immune responses, and interaction with coinhibitory receptors attenuates immune responses. There are multiple known members of the family, including but not limited to B7.1 (CD80), B7.2 (CD86), inducible costimulator ligand (ICOS-L), programmed death-1 ligand (PD-L1), programmed death-2 ligand (PD-L2), B7-H3, and B7-H4. While not being bound a particular theory, the present invention contemplates directing immunosuppressant B7 family members or derivatives thereof to the target site of abnormal immune system response in a person in need thereof, such that the abnormal immune system response is suppressed. An effective suppression of abnormal immune system response represents the treatment of auto-immune or inflammatory diseases.

Programmed death-ligand 1 (PD-L1) is a 40 kDa type 1 transmembrane protein that has been speculated to play a major role in suppressing the immune system during particular events such as pregnancy, tissue allografts, auto-immune disease and other disease states such as hepatitis. Normally the immune system reacts to foreign antigens where there is some accumulation in the lymph nodes or spleen which triggers a proliferation of antigen-specific CD8+ T cell. The formation of PD-1 receptor/PD-L1 or B7.1 receptor/PD-L1 ligand complex transmits an inhibitory signal which reduces the proliferation of these CD8+ T cells at the lymph nodes and supplementary to that PD-1 is also able to control the accumulation of foreign antigen specific T cells in the lymph nodes through apoptosis which is further mediated by a lower regulation of the gene Bcl-2. PD-L1 binds to its receptor, PD-1, found on activated T cells, B cells, and myeloid cells, to modulate activation or inhibition. The affinity between PD-L1 and PD-1, as defined by the dissociation constant Kd, is 770 nM.

PD-L1 also has an appreciable affinity for the co-stimulatory molecule CD80 (B7-1), but not CD86 (B7-2). CD80's affinity for PD-L1, 1.4 μM, is intermediate between its affinities for CD28 and CTLA-4 (4.0 μM and 400 nM, respectively). The related molecule PD-L2 has no such affinity for CD80 or CD86, but shares PD-1 as a receptor (with a stronger Kd of 140 nM). It has been shown that PD-1, up-regulated on activated CD4 T-cells, can bind to PD-L1 expressed on monocytes and induces IL-10 production by the latter.

PD-1/PD-L1 interaction is implicated in auto-immunity from several lines of evidence. NOD mice, an animal model for auto-immunity in that they exhibit a susceptibility to spontaneous development of type I diabetes and other auto-immune diseases, have been shown to have precipitated onset of diabetes from blockade of PD-1 or PD-L1 (but not PD-L2).

In humans, PD-L1 was found to have altered expression in pediatric patients with systemic lupus erythematosus (SLE). Studying isolated peripheral blood mononuclear cell (PBMC) from healthy children, immature myeloid dendritic cells and monocytes expressed little PD-L1 at initial isolation, but spontaneously up-regulated PD-L1 by 24 hours. In contrast, both mDC and monocytes from patients with active SLE failed to upregulate PD-L1 over a 5 day time course, expressing this protein only during disease remissions. This suggests one mechanism whereby peripheral tolerance is lost in SLE. While not being bound to a particular theory, it is believed that PD-L1 and other B7 proteins or derivatives thereof are useful in the treatment of auto-immune or inflammatory diseases because they act as selective immunosuppressants.

The present invention provides a method for treating auto-immune or inflammatory diseases through the administration of B7 family proteins or derivatives thereof. In some embodiments, a treatment is directed to an auto-immune and inflammatory skin disease, such as vitiligo, psoriasis, or atopic dermatitis. In some embodiments, the B7 family protein comprises programmed death ligand-1 or derivative thereof. In some embodiments, the B7 family proteins or derivatives thereof are administered topically.

In some embodiments, the present invention comprises administering PD-L1 to a subject in need thereof to treat an auto-immune or inflammatory disease through inhibiting the subject's immune system response. While not being bound to a particular theory, it is believed that by PD-L1 may bind to PD-1 receptors found on activated T-cells, B-cells, and myeloid cells to inhibiting their activity, while also binding to co-stimulatory B7 variants, down-regulating their ability to stimulate immune cell activity.

In some embodiments, the present invention is directed to methods of administering modified B7 family proteins that offer enhanced therapeutic efficacy. Suitable modifications include any of a number of techniques known to those of skill in the art to alter various factors affecting therapeutic efficacy. For example, pegylation is a modification wherein a polyethylene glycol (PEG) polymer chain is covalently attached to a protein or other molecule. This modification can reduce immunogenicity and antigenicity and increase the hydrodynamic size of a protein, thereby prolonging its circulatory time and reducing renal clearance. These alterations may increase the efficacy of a therapeutic protein, such that a pegylated B7 protein may be more effective in treating a blood-borne auto-immune disease than an unpegylated B7 protein.

Another such protein modification is palmitoylation, a covalent attachment of fatty acids such as palmitic acid to proteins. This modification typically enhances the hydrophobicity of proteins and contributes to their membrane association. Other known post-translational protein modifications that may affect therapeutic efficacy include, but are not limited to, acetylation, formylation, myristoylation, amidation, detyrosination, glycosylation, dephosphorylation, succinimide formulation, methylation, transglutimination, ubiquitination, sumoylation, citrullination, hydroxylation, diphthamide formation, c-mannosylation, proteolysis, crosslink formation between two to four amino acids, or chromophore formation. While not being bound to a particular theory, it is believed that similar modification to B7 family proteins or derivatives thereof may be used to increase their therapeutic efficacy in the treatment of auto-immune or inflammatory diseases.

Protein modification may also occur before the protein is created by altering the genetic code programming used in its construction. For instance, artificially splicing genetic code which programs for one protein with genetic code for another protein using recombinant DNA technology can result in a recombinant fusion protein. While not being bound to any particular theory, it is believed that genetic modification to an organism used to produce naturally-occurring B7 family proteins could produce variants or derivatives of B7 family proteins with increased therapeutic efficacy in the treatment of auto-immune or inflammatory diseases.

The administration of B7 family proteins or derivatives thereof may be accomplished in any methods of administration, alone or with acceptable excipients and delivery methods, known in the art. The dosage of the compound administered will depend on the condition being treated, the particular compound administered, and other clinical factors, such as the weight and condition of the human or animal to be treated and the route of administration of the compound. It is to be understood that the present invention has application for both human and veterinary use. For other routes of administration, the concentration of B7 family protein or derivative thereof in the delivery vehicle is adjusted to result in blood levels of the B7 family protein or derivative thereof that are roughly equivalent to the blood level resulting from the intravenous administration.

The formulations of present include those suitable for oral, rectal, ophthalmic, (including intravitreal or intracameral) nasal, topical (including buccal and sublingual), vaginal and parenteral (including subcutaneous, intramuscular, intravenous, intradermal, intratracheal, and epidural) administration of B7 family proteins or derivatives thereof. The formulations may conveniently be presented in unit dosage form and may be prepared by conventional pharmaceutical techniques. (See, for example. Pharmaceutical Dosage Forms and Drug Delivery Systems, 6th Edition, Ansel, H.C. et al., Ed, Williams & Wilkens, 1995, which is incorporated herein by reference.) Such techniques include the step of bringing into association the active ingredient (i.e., the B7 family protein or derivative thereof) and pharmaceutical carriers or excipients. In general, the formulations are prepared by uniformly and intimately bringing into association the active ingredient with liquid carriers or finely divided solid carriers or both, and then, if necessary, shaping the product. Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier. Formulations suitable for topical administration to the skin may be presented as ointments, creams, gels and pastes comprising the active ingredient to be administered in a pharmaceutical acceptable carrier. Topical delivery systems include a transdermal patch and a transdermal gel containing the ingredient to be administered.

Formulations for rectal administration may be presented as a suppository with a suitable base comprising, for example, cocoa butter or a salicylate. Formulations suitable for nasal administration, wherein the carrier is a solid, include a coarse powder having a particle size, for example, in the range of 20 to 500 microns, which is administered in the manner in which snuff is administered, i.e., by rapid inhalation through the nasal passage from a container of the powder held close up to the nose. Suitable formulations for nasal administration, wherein the carrier is a liquid, as for example, a nasal spray or nasal drops, include aqueous or oily solutions of the active ingredient. Formulations suitable for vaginal administration may be presented as pessaries, tampons, creams, gels, pastes, foams or sprays containing in addition to the active ingredient such carriers as are known in the art to be appropriate.

Formulations suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in freeze-dried (lyophilized) conditions requiring only the addition of the sterile liquid carrier, for example, water for injections, immediately prior to use. Extemporaneous injection solutions and suspensions may be prepared from sterile powders, granules, and tablets of the kind previously described.

The pharmaceutical compositions described herein can, if desired, include one or more pharmaceutically acceptable excipients. The term “excipient” herein means any substance, not itself a therapeutic agent, used as a carrier or vehicle for delivery of a therapeutic agent to a subject or added to a pharmaceutical composition, for example, to improve its handling or storage properties or to permit or facilitate formation of a dose unit of the composition. Excipients include, by way of illustration and not limitation, solvents, thickening agents, penetration enhancers, wetting agents, lubricants, emollients, substances added to mask or counteract a disagreeable odor, fragrances, and substances added to improve appearance or texture of the composition. Any such excipients can be used in any dosage forms of according to the present disclosure. The foregoing list of excipients is not meant to be exhaustive but merely illustrative as a person of ordinary skill in the art would recognize that additional excipients could be used to achieve the desired goals for delivery of the B7 family protein or derivative thereof.

Compositions of the disclosure containing excipients can be prepared by any technique known to a person of ordinary skill in the art of pharmacy, pharmaceutics, drug delivery, pharmacokinetics, medicine or other related discipline that comprises admixing an excipient with a drug or therapeutic agent. Known administration methods allow for routine modification to ensure delivery of active ingredients, such as the B7 family proteins or derivatives thereof, to the site of their activity within the body. For instance, enteric coatings on an oral tablet formulation may ensure the active ingredient is protected from the harmful acids in the stomach, but released once passed to the small intestine. Similarly, a topical cream may be formulated with polyphenols to prevent UVB-induced oxidation of suspended proteins, preventing their degradation and maintaining therapeutic efficacy and prolonging shelf life. In some embodiments, B7 family proteins are co-administered with one or more immunosuppresants including, but not limited to, cyclosporine, rapamycin, azathioprine, basiliximab, prednisone, and combinations thereof.

Some embodiments of the invention are directed to a method of treating auto-immune or inflammatory skin disease through topical administration of B7 family proteins or derivatives thereof. Such auto-immune skin diseases include conditions like vitiligo, wherein pigment-producing melanocytes, located in the stratum basale of the epidermis, or the melanin they produce are attacked by the body's own immune system. While not being bound to any particular theory, it is believed that introducing immunosuppressant B7 family proteins to the melanocytes and surrounding tissue would suppress T-cell, B-cell, or other immune system response to those areas. Topical administration in this instance may limit immunosuppression to the site of abnormal immune activity, here the skin, while preventing generalized immunosuppression, allowing normal immune system response to protect the rest of the body's systems. These embodiments of the invention may include pharmaceutical or cosmeceutical creams, salves, lotions, powders, suspensions, or other appropriate vehicles for application to the skin.

The present invention has been described with reference to certain exemplary embodiments thereof. However, it will be readily apparent to those skilled in the art that it is possible to embody the invention in specific forms other than those of the exemplary embodiments described above. This may be done without departing from the spirit and scope of the invention. The exemplary embodiments is are merely illustrative and should not be considered restrictive in any way. The scope of the invention is defined by the appended claims and their equivalents, rather than by the preceding description.

All United States patents and applications, foreign patents, and publications discussed above are hereby incorporated herein by reference in their entireties.

We claim:

1. A method of treating an auto-immune or inflammatory disease comprising administering an amount of a B7 family protein or derivative thereof to a subject in need thereof.

2. The method of claim 1, wherein the auto-immune or inflammatory disease is an auto-immune or inflammatory skin disease.

3. The method of claim 2, wherein the auto-immune or inflammatory skin disease is psoriasis, vitiligo, atopic dermatitis, epidermolysis bullosa acquista, pemphigus vulgaris, IgA-mediated bullous dermatoses, systemic lupus erythematosus, alopecia areata, porphyria, or scleroderma.

4. The method of claim 1, wherein the B7 family protein comprises programmed death ligand-1 or derivatives thereof.

5. The method of claim 1, wherein the B7 family protein or derivative thereof is administered topically.

6. The method of claim 1, wherein the B7 family protein derivative comprises a peptide fragment.
